(19) United States
(12) Patent Application Publication
(10) Pub. No.: US 2023/0181811 A1
(43) Pub. Date: Jun. 15, 2023

(54) MEDICAL SUCTION DEVICE UTILIZING VACUUM ACCUMULATION

(71) Applicant: AEROBIOTIX, LLC, Miamisburg, OH (US)

(72) Inventor: David Louis Kirschman, Dayton, OH (US)

(73) Assignee: AEROBIOTIX, LLC, Miamisburg, OH (US)

(21) Appl. No.: 18/062,645

(22) Filed: Dec. 7, 2022

Related U.S. Application Data
(60) Provisional application No. 63/289,813, filed on Dec. 15, 2021.

(51) Int. Cl. A61M 1/00

(52) U.S. Cl. CPC A61M 1/74; A61M 1/743; A61M 1/81; A61M 1/79; A61M 1/73; A61B 2218/007; A61M 2205/82; A61M 2205/3344; A61M 2205/3337

(57) ABSTRACT

A suction device and system utilizing a moveable and/or portable dry vacuum accumulator for vacuum accumulation. The device has at least one accumulator or reservoir for storing a vacuum charge. The device is initially charged by a vacuum source, such as a vacuum from a wall vacuum source as is conventional in a hospital. The vacuum may be boosted using a vacuum boosting pump that may be pneumatic, hydraulic or even electric. The device can be charged to a second predetermined vacuum level which can be different from both atmospheric pressure as well as a first predetermined vacuum level supplied by the vacuum source. Apparatus is also provided for boosting vacuum or for increasing or decreasing a volume in the at least one accumulator or reservoir.

### CROSS-REFERENCE TO RELATED APPLICATION

The present application claims priority to provisional U.S. Application Ser. No. 63/289,813 filed Dec. 15, 2021, to which Applicant claims the benefit of the filing date of this provisional application, which application is incorporated herein by reference and made a part hereof.

### BACKGROUND OF THE INVENTION

### 1. Field of the Invention

This invention relates to a medical suction device that utilizes a vacuum accumulator and that is adapted to store a vacuum for use by a user, such as a surgeon, during a surgical procedure. The medical suction device may be coupled to and disconnected from a vacuum source and is portable so that it can provide a predetermined vacuum level to a surgical instrument.

### 2. Description of the Related Art

Devices for the suction of fluids, gases, and airborne particulates are commonly used in medical settings, such as hospital or care rooms or surgery rooms. Infectious aerosols and surgical smoke arising from tissue cauterization are of particular concern for the safety of patients and healthcare personnel. Traditionally, hospital rooms and operating suites have built-in wall suction outlets and valves. These outlets and valves are attached to various tubing and suction tip means to evacuate contaminants at the site of the patient. However, the negative airflow provided by typical wall suction is oftentimes not adequate to effectively evacuate heavy contamination loads. As such, hospitals have employed self-contained portable vacuum devices for this purpose. These devices have the benefit of high airflow capacity, however they tend to be noisy, expensive, space occupying, and disruptive of healthcare workflows.

What is needed, therefore, is a device which effectively increases the airflow and negative pressure arising from existing wall suction systems. Because suction needs are typically intermittent, the continuous vacuum provided by wall suction can be stored in an accumulator system which will generate a progressively negative pressure or vacuum while not in use. The system will further incorporate a valve means whereby a user can generate intermittent bursts of high vacuum flows via the accumulator. This invention further comprises various filtration, control, and safety means.

### SUMMARY OF THE INVENTION

One object of the invention is to provide a vacuum accumulator adapted to store a vacuum for use by a user, such as a surgeon.

Another object of the invention is to provide a vacuum accumulator that is portable and can be used in different environments such as a hospital room, surgical center or other room or facility where a vacuum is desired.

Another object of the invention is to provide a vacuum accumulator that can provide intermittent and boosts of vacuum that are higher than a vacuum source used to charge the vacuum accumulator, such as is provided in a hospital room or surgical center that has a wall vacuum outlet.

Yet another object of the invention is to provide a vacuum accumulator that can provide intermittent boosts of vacuum to a surgical instrument in response to a user actuating a control, such as foot switch or a switch on the surgical instrument.

Yet another object of the invention is to provide a vacuum accumulator having at least one input and at least one output for charging the vacuum accumulator and for permitting the vacuum to supply a vacuum to at least one or a plurality of surgical instruments or uses.

Still another object of the invention is to provide a portable vacuum accumulator that can store a vacuum at a higher level than what is provided by a vacuum source.

Another object of the invention is to provide a vacuum accumulator device that comprises a vacuum boost.

Another object of the invention is to provide a vacuum accumulator device that comprises a vacuum volume that can be increased or decreased in order to increase or decrease, respectively, a vacuum in the accumulator or reservoir.

Another object of the invention is to provide a portable vacuum accumulator device that has decontamination or filtration means adapted to prevent the vacuum accumulator device from receiving fluids or contamination.

Another object is to provide a vacuum accumulator device that is a dry vacuum and in some embodiments that is also portable.

In one aspect, one embodiment of the invention comprises a medical suction device for use in a room, the medical suction device comprising a vacuum accumulator that defines at least one vacuum accumulator or reservoir in fluid communication with at least one inlet port for coupling to a vacuum source and at least one outlet port for coupling to an instrument that utilizes a vacuum, the vacuum source providing a vacuum at a first predetermined vacuum level; at least one control or control means for controlling an operation of the medical suction device; the medical suction device being adapted to store a vacuum charge in the at least one vacuum accumulator or reservoir so that the at least one vacuum accumulator or reservoir may be disconnected from the vacuum source and still maintain at least a portion of the first predetermined vacuum level which may be utilized by the instrument.

In another aspect, another embodiment of the invention comprises a medical suction device comprising an external connection to a vacuum source; a vacuum accumulator; a user selectable suction switch; at least one suction output port; and a control means; wherein the medical suction device receives a continuous low-flow vacuum input and transmits an intermittent high-flow vacuum output; the intermittent high-flow vacuum output initiated by user activation of the switch.

In one aspect, one embodiment of the invention comprises a method for providing medical suction comprising: connecting an accumulator to a constant low-flow vacuum source; evacuating air from the accumulator; selectively actuating a release valve on the accumulator thus creating an intermittent vacuum-powered airflow of greater volume (for instance as measured in cubic feet per minute) than a constant low-flow volume arising from the vacuum source; and directing an intermittent high-flow vacuum to an area of need.

This invention, including all embodiments shown and described herein, could be used alone or together and/or in combination with one or more of the features covered by one or more of the following list of features which may be combined, used independently or the like:

The medical suction device wherein the at least one vacuum accumulator or reservoir is portable and moveable when the at least one inlet port is coupled to the vacuum source and when it is disconnected therefrom while maintaining a vacuum at a second predetermined vacuum level.

The medical suction device wherein the second predetermined vacuum level is less negative than the first predetermined vacuum level.

The medical suction device wherein the second predetermined vacuum level is more negative than the first predetermined vacuum level.

The medical suction device wherein an airflow rate at the at least one outlet port is greater than an airflow rate at the at least one inlet.

The medical suction device wherein the first predetermined vacuum level is substantially the same as or less negative than the second predetermined vacuum level while the airflow rate at the instrument is greater than an airflow rate at the at least one outlet port.

The medical suction device wherein the first predetermined vacuum level is different than the second predetermined vacuum level while the airflow rate at the instrument is greater than an airflow rate at the at least one outlet port.

The medical suction device wherein the at least one control means or control comprises at least one of a switch, a foot switch or actuator, the airflow rate being constant, intermittent or timed out in response to a user actuating the at least one switch, foot switch or actuator.

The medical suction device wherein the medical suction device comprises at least one coupler for detachably coupling the at least one vacuum accumulator or reservoir to the vacuum source and providing fluid communication between the at least one vacuum accumulator or reservoir and the vacuum source so that a pressure lower than atmospheric pressure can be maintained in the at least one vacuum accumulator or reservoir when the at least one vacuum accumulator or reservoir is not coupled to the vacuum source.

The medical suction device wherein the at least one coupler comprises at least one of a check valve or pressure regulator.

The medical suction device wherein the at least one coupler is adapted to allow for disconnection of the medical suction device from the vacuum source after the at least one vacuum accumulator or reservoir is charged with a vacuum and while retaining the vacuum in the at least one vacuum accumulator or reservoir, the at least one coupler allowing the medical suction device to be portably transported while holding a vacuum in the at least one vacuum accumulator or reservoir in order to enable the medical suction device to supply a vacuum without an external connection to the vacuum source.

The medical suction device wherein the at least one vacuum accumulator or reservoir is adapted to store a predetermined amount of negative pressure after being coupled to and then disconnected from the vacuum source, the at least one vacuum accumulator or reservoir being transportable.

The medical suction device wherein the at least one vacuum accumulator or reservoir comprises a plurality of wheels for supporting the at least one vacuum accumulator or reservoir and for enabling it to be moved, portable or transported in the room.

The medical suction device wherein the vacuum source provides a substantially continuous low-flow vacuum to the at least one vacuum accumulator or reservoir, the medical suction device comprising at least one vacuum booster for boosting the substantially continuous low-flow vacuum from the first predetermined vacuum level to a second predetermined vacuum level, the first predetermined vacuum level being lower than the second predetermined vacuum level.

The medical suction device wherein the at least one vacuum booster further comprises an internal powered or manual vacuum pump for the medical suction device to be used without an external vacuum connection to the vacuum source or when the vacuum source is not sufficient to generate desired vacuum levels in the at least one vacuum accumulator or reservoir.

The medical suction device wherein the at least one vacuum booster comprises a pump operatively situated in or on the medical suction device for creating or increasing a negative pressure in the at least one vacuum accumulator or reservoir.

The medical suction device wherein the at least one vacuum booster comprises a piston coupled to a piston drive for driving the piston inside the at least one vacuum accumulator or reservoir, the piston being driven by the piston drive in order to change a volume in the at least one vacuum accumulator or reservoir in order to increase a negative pressure in the at least one vacuum accumulator or reservoir.

The medical suction device wherein the at least one vacuum booster comprises a pump operatively coupled to the at least one vacuum accumulator or reservoir for increasing a negative pressure in the at least one vacuum accumulator or reservoir.

The medical suction device wherein the vacuum source is external to the medical suction device and the vacuum charge from the vacuum source creates a contained pressure environment at the second predetermined level in the at least one vacuum accumulator or reservoir that is lower than atmospheric pressure, the at least one vacuum accumulator or reservoir comprising the at least one inlet port for coupling to the vacuum source and the at least one outlet port for coupling for supplying vacuum to the instrument.

The medical suction device wherein the at least one vacuum accumulator or reservoir comprises at least one leak resistant container of metallic or polymer construction such that there is a fixed pressure and volume relationship within the at least one vacuum accumulator or reservoir, the at least one vacuum accumulator or reservoir comprising at least one of a cylinder or hollow body adapted to define the at least one vacuum accumulator or reservoir.

The medical suction device wherein the at least one vacuum accumulator or reservoir comprises a leak-resistant, variable-volume container adapted to define a predetermined pressure-volume relationship in the at least one vacuum accumulator or reservoir.

The medical suction device wherein the leak-resistant, variable-volume container comprises at least one of a flexible membrane, a flexible bladder, or a moveable piston adapted to vary a volume within the at least one vacuum accumulator or reservoir while retaining a vacuum in the at least one vacuum accumulator or reservoir at a third predetermined vacuum level.

The medical suction device wherein the third predetermined vacuum level is greater than both the first predetermined vacuum level and the second predetermined vacuum level.

The medical suction device wherein the medical suction device comprises at least one decontamination means or decontaminator for decontaminating to facilitate decontaminating the at least one vacuum accumulator or reservoir in order to prevent survival and growth of microorganisms within the at least one vacuum accumulator or reservoir or the medical suction device.

The medical suction device wherein the at least one control or control means comprises a switch having at least one valve situated between the at least one vacuum accumulator or reservoir and the at least one inlet port and adapted to open or close in response to a user input.

The medical suction device wherein the user input may comprise a foot pedal, manual switch, current-activated switch or a triggering in response to the instrument or external medical equipment.

The medical suction device wherein the external medical equipment is an electrosurgery device.

The medical suction device wherein the at least one control or control means comprises timer to provide suction bursts of a user-selected duration.

The medical suction device wherein the at least one outlet port is adapted and dimensioned to receive various medical and surgical effector couplings, the medical and surgical effector couplings comprising at least one of a medical tube, a suction tip, an electrosurgical pencil, a smoke evacuator device, a laparoscopic device, a cautery or a surgical device that requires operable suction.

The medical suction device wherein the at least one control or control means comprises at least one user interface for controlling at least one of suction activation, suction timing, accumulator pressure, input pressure or output pressure.

The medical suction device wherein the medical suction device comprises at least one of at least one valve for controlling a vacuum level in the at least one vacuum accumulator or reservoir, at least one pressure sensor for sensing a pressure in the at least one vacuum accumulator or reservoir, or at least one velocity sensor for sensing a velocity of airflow into or out of the at least one vacuum accumulator or reservoir.

The medical suction device wherein the medical suction device further comprises at least one filter or filter filtration means for removing contamination and organisms into or out of the at least one vacuum accumulator or reservoir from an airflow.

The medical suction device wherein the at least one filter or filter filtration means comprises at least one of mechanical, absorptive, catalytic or chemical filtration capacities, including ULPA (ultra-low penetration air) filtration, HEPA (high efficiency particulate air) filtration, carbon filtration, catalytic filtration, pleated media filtration, solid media filtration, ultraviolet germicidal irradiation, and similar means for removing airborne contamination and organisms.

The medical suction device wherein the at least one vacuum accumulator or reservoir is a dry vacuum and does not collect fluids when the instrument is being used by a user.

The medical suction device wherein the medical suction device comprises a fluid trap situated between the at least one vacuum accumulator or reservoir and the instrument in order to trap fluids prior to entering the medical suction device so that the at least one vacuum accumulator or reservoir is a dry vacuum accumulator.

The medical suction device wherein the instrument collects or generates vapor or fluids during a medical procedure and in the at least one vacuum accumulator or reservoir and provides the vacuum to the instrument in order to collect fluids, smoke, vapor and/or debris and the like during the medical procedure; the medical suction device further comprising a fluid trap for preventing the fluids, smoke, vapor and/or debris or contamination from entering the at least one vacuum accumulator or reservoir.

The medical suction device wherein the external connection comprises a hose or piping means to attach to an external vacuum source; the external connection communicates an external vacuum charge with the vacuum accumulator.

The medical suction device wherein the external connection means further comprising a one way check valve and/or pressure regulator; the check valve to allow for disconnection of the suction device from the vacuum source after the vacuum charge while retaining internal accumulator vacuum; the disconnection and retention allowing the suction device to be portably transported and used without an external connection.

The medical suction device wherein the vacuum accumulator receiving a vacuum charge from the external connection to create a contained pressure environment lower than atmospheric pressure; the accumulator comprising a vacuum input and output means.

The medical suction device wherein the vacuum accumulator comprising a rigid, leak-resistant container of metallic or polymer construction such that there is a fixed pressure/volume relationship within the accumulator; the accumulator comprising a cylinder or other hollow body.

The medical suction device wherein the vacuum accumulator comprising a non-rigid, leak-resistant, variable-volume container of metallic or polymer construction such that there is a variable pressure/volume relationship within the accumulator; the variable-volume means comprising flexible membrane, mobile piston or similar means to vary accumulator volume, while retaining a vacuum seal; the variable-volume means providing a benefit of increased vacuum capacity and/or negative pressure levels.

The medical suction device wherein the vacuum accumulator further comprising an internal decontamination means consisting of ultraviolet irradiation, catalytic oxidation, electrostatic generation, ion generation, ozone generation, or similar means to prevent survival and growth of microorganisms within the accumulator and or suction device.

The medical suction device wherein the suction switch comprising a valve between the accumulator and the at least one suction port; the valve comprising an electromagnetic solenoid or similar mechanically actuated design, providing near instantaneous opening of the valve in response to a user input; the input may comprise a foot pedal, manual switch, current-actuated switch, or triggering from external medical equipment, such as an electrosurgery device; the valve may optionally include a timer means to provide suction bursts of a user-selected duration.

The medical suction device wherein the at least one suction port dimensioned to receive various medical and surgical effector ends such as medical tubing, suction tips, electrosurgical pencils, smoke evacuation devices, laparoscopic devices, and other means which require operable suction.

The medical suction device wherein the control means comprising a user interface controlling one or more of suction activation, suction timing, accumulator pressure, input pressure and output pressure; the interface in communication with corresponding valves, pressure, and/or velocity sensors.

The medical suction device wherein the medical suction device and/or the vacuum accumulator comprising a filtration means, with at least one of mechanical, absorptive, catalytic or chemical filtration capacities, including ULPA filtration, HEPA filtration, carbon filtration, catalytic filtration, pleated media filtration, solid media filtration, ultraviolet germicidal irradiation, and similar means for removing airborne contamination and organisms.

The medical suction device wherein the medical suction device optionally comprising an internal powered or manual vacuum pump to allow for the device to be used without an external vacuum connection, or when the external vacuum source is not sufficient to generate adequate accumulator vacuum levels.

These and other objects and advantages of the invention will be apparent from the following description, the accompanying drawings and the appended claims.

BRIEF DESCRIPTION OF THE ACCOMPANYING DRAWINGS

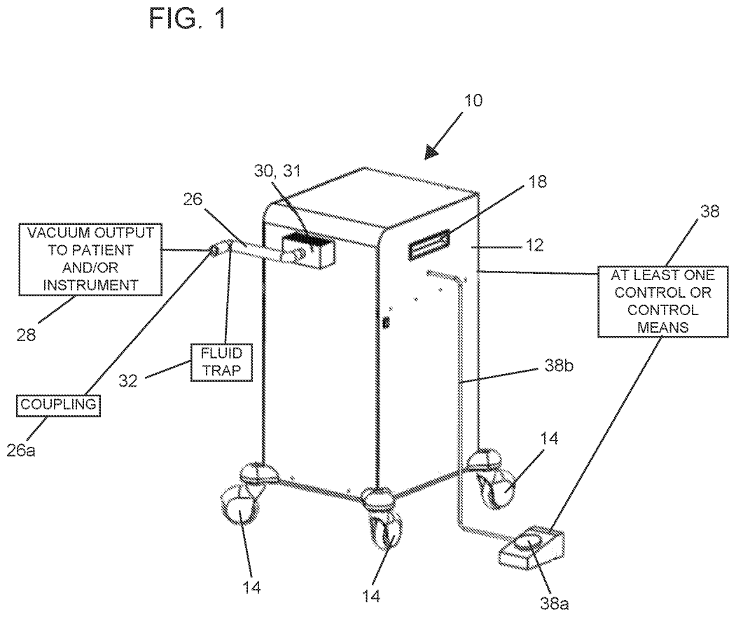

FIG. 1 is a front view of one embodiment of a medical suction device in accordance with one embodiment of the invention;

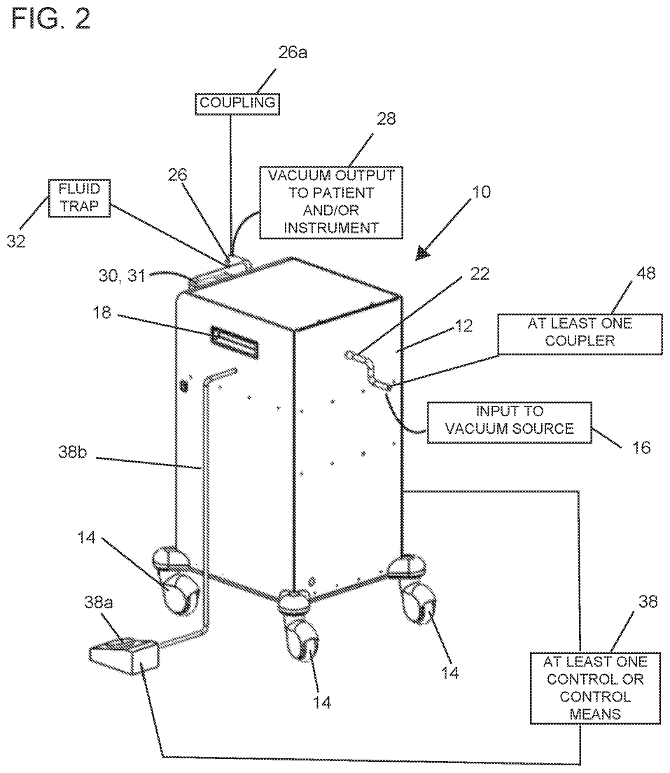

FIG. 2 is a perspective view showing a rear view of the embodiment shown in FIG. 1;

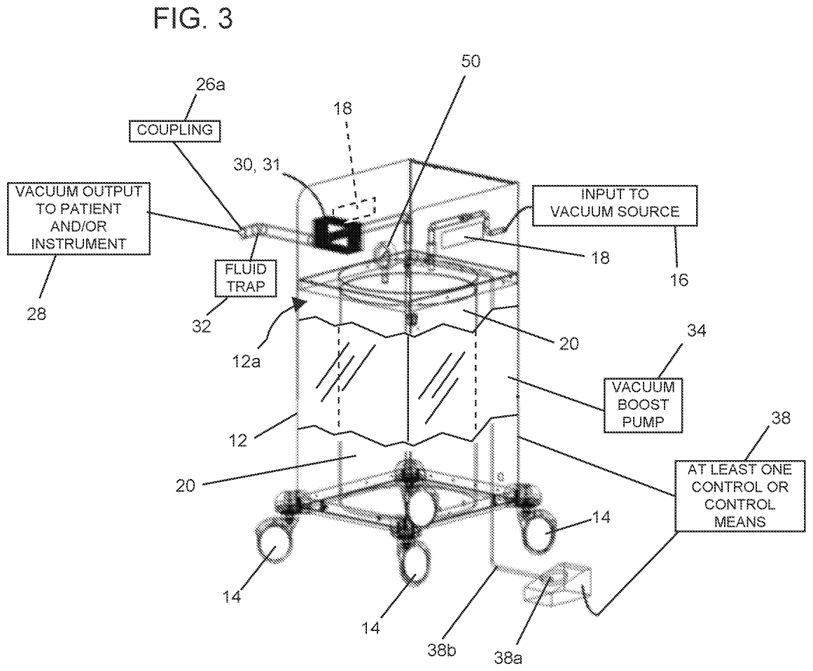

FIG. 3 is partial fragmentary view showing details of various components of the embodiment shown in FIGS. 1 and 2;

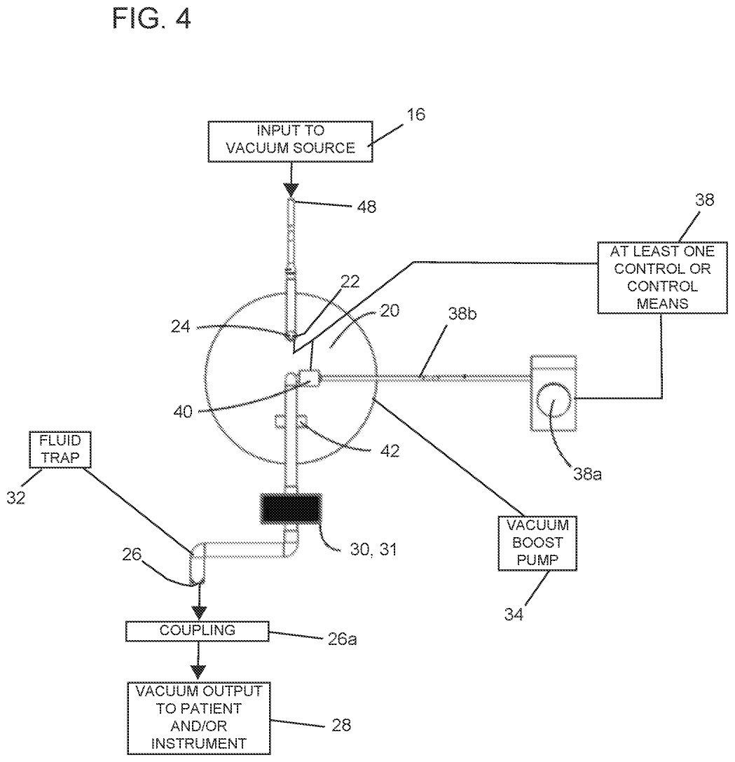

FIG. 4 is schematic top view the device shown in FIGS. 1-3 further illustrating various components of the embodiment shown in FIG. 1;

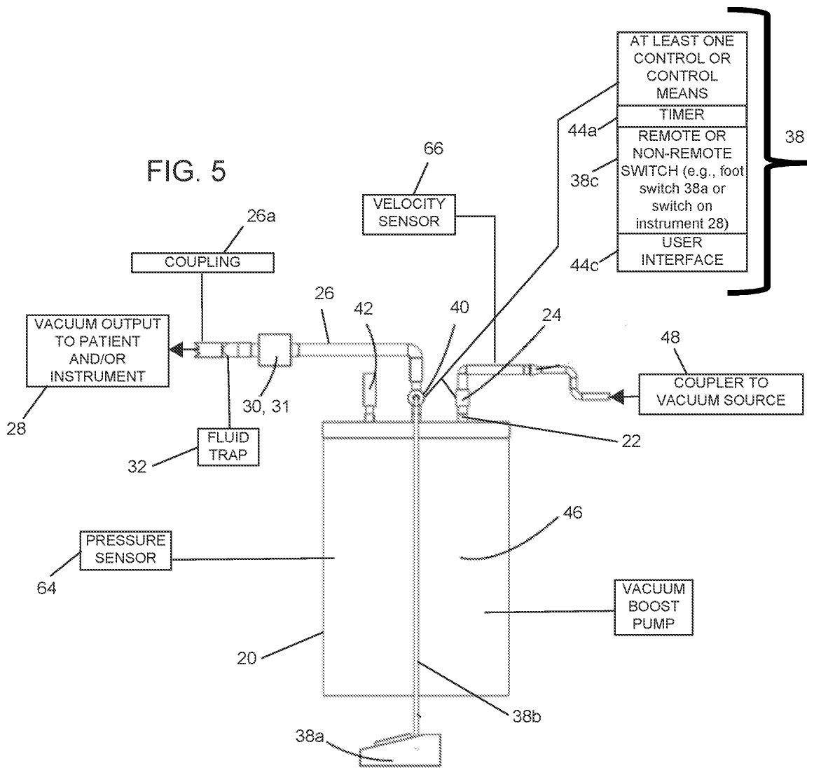

FIG. 5 is another view of the at least one vacuum accumulator or reservoir outside the housing of the device shown in FIGS. 1 and 2;

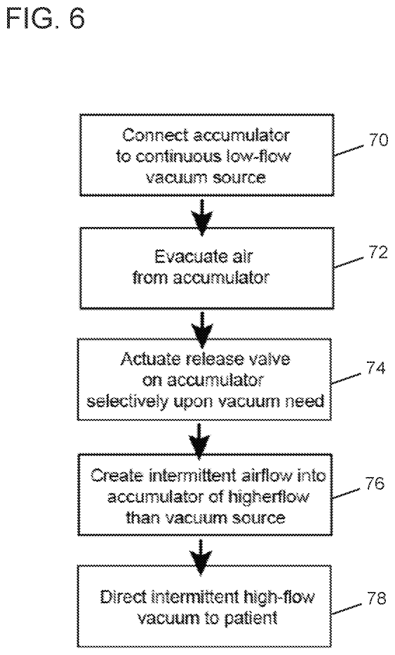

FIG. 6 is schematic flow chart illustrating a method in accordance with one embodiment of the invention; and

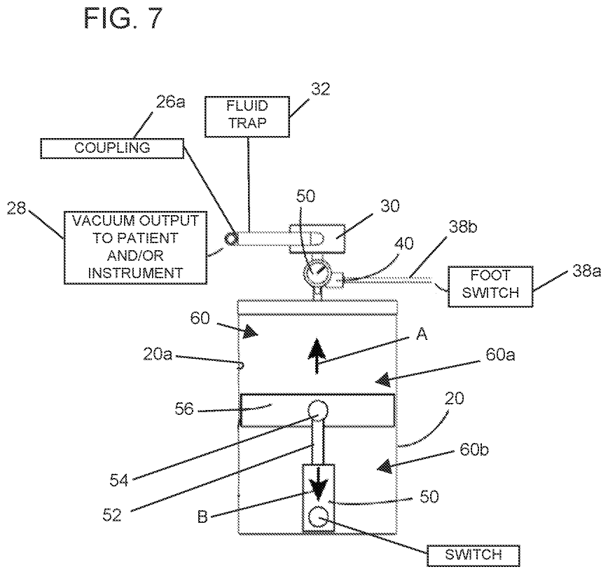

FIG. 7 is another embodiment illustrating a variable-volume accumulator to increase vacuum pressure and/or capacity.

### DETAILED DESCRIPTION OF THE PREFERRED EMBODIMENTS

Referring now to FIG. 1, a medical suction device 10 is shown. The medical suction device 10 is adapted to effectively accumulate and increase an airflow and negative pressure arising from, for example, existing wall suction systems, such as those systems in a hospital room or operating room. The medical suction device 10 is particularly adapted for being transportable and for providing a continuous vacuum or negative pressure and storing the vacuum both during and while not in use.

The medical suction device 10 comprises a housing 12 and a plurality of wheels 14, such as castor wheels, mounted thereon and which are adapted to make the device mobile and portable within the room. As will become apparent later herein, the medical suction device 10 is adapted to be coupled to a suction or vacuum source, such as a built-in wall suction outlet or vacuum source 16 (FIG. 2), which are common in hospital rooms and operating suites. The housing 12 comprises a plurality of handles 18 for gripping the medical suction device 10 and for moving it as desired.

Internally situated and mounted in the housing 12 is at least one vacuum accumulator or reservoir 20, which is best illustrated in the partial fragmentary view of FIG. 3 and FIGS. 4-5 and which will be described in more detail later herein. The at least one vacuum accumulator or reservoir 20 may be of metallic or polymer construction and is adapted to store and hold a negative pressure or pressure lower than atmospheric pressure and to be situated in the room during use.

As will be described in greater detail later herein, the medical suction device 10 comprises at least one inlet port or passageway 22 for providing fluid communication and coupling between the vacuum source 16 and the at least one vacuum accumulator or reservoir 20, which in the illustration being described is a built-in wall suction outlet or vacuum source 16, that provides a negative pressure to the room or operating suite. The at least one inlet port or passageway 22 comprises a check valve or pressure regulator 24 adapted to permit and maintain a negative pressure and evacuation from the at least one vacuum accumulator or reservoir 20 while preventing a positive pressure from entering the at least one vacuum accumulator or reservoir 20.

The at least one vacuum accumulator or reservoir 20 further comprises at least one outlet port or passageway 26 for providing fluid communication and a vacuum to a medical or surgical instrument or effector 28 so that a negative pressure may be applied thereto. The at least one outlet port or passageway 26 comprises a coupling 26a for providing a fluid-tight coupling to the medical or surgical instrument or effector 28. It should be understood that the at least one outlet port or passageway 26 is adapted and dimensioned and comprises a coupling that is adapted to receive various medical and surgical instrument or effectors 28 which are conventionally known. Such medical and surgical instruments or effectors 28 may comprise at least one of a medical tube, a suction tip, an electrosurgical pencil, a smoke evacuator device, a laparoscopic device, a cautery device or a surgical device that requires operable suction. In the illustration being described, the medical or surgical instrument or effector 28 may be external medical equipment, such as an electrosurgery or electrocautery device. Thus, the at least one outlet port or passageway 26 and coupling 26a is adapted and dimensioned to receive various medical and surgical effector couplings (not shown) comprising at least one of the medical tube, suction tip, electrosurgical pencil, smoke evacuator device, laparoscopic device, cautery device or the surgical device that requires operable suction as mentioned.

As will become apparent, the negative pressure in the at least one vacuum accumulator or reservoir 20 is adapted and used to provide a negative pressure or vacuum through the at least one outlet port or passageway 26 and to the medical and surgical instrument or effector 28, which is generally associated with a patient during a surgical procedure.

Note that the at least one outlet port or passageway 26, which is the airflow inlet into the at least one vacuum accumulator or reservoir 20, comprises at least one filter or filtration means 30 for filtering the fluid or airstream as it passes through the at least one outlet port or passageway 26 and before it enters the at least one vacuum accumulator or reservoir 20. It should be appreciated that the at least one vacuum accumulator or reservoir 20 is a “dry” accumulator and does not collect liquids. In one embodiment, the medical suction device 10 may comprise at least one fluid trap 32 which collects and traps fluids and debris prior to entering the at least one vacuum accumulator or reservoir 20. In this regard, the at least one fluid trap 32 may be a conventional gravity fluid trap (not shown) coupled to and in fluid communication with the at least one outlet port or passageway 26. In this regard, the at least one fluid trap 32 may be upstream of the at least one filter or filtration means 30 or the at least one fluid trap 32 may be downstream thereof as illustrated in FIGS. 3 and 5, respectively. Advantageously, the at least one fluid trap 32 and the at least one filter or filtration means 30 are adapted to trap fluid, unwanted debris and contamination prior to entering the at least one vacuum accumulator or reservoir 20 so that the at least one vacuum accumulator or reservoir 20 does not get contaminated and remains dry.

In the illustration being described, the vacuum source 16 provides a vacuum at the first predetermined vacuum level as mentioned. Once the at least one vacuum accumulator or reservoir 20 is coupled to the vacuum source 16 via the at least one inlet port or passageway 22, the at least one vacuum accumulator or reservoir 20 is adapted to store a vacuum charge in the at least one vacuum accumulator or reservoir 20. Note that the at least one vacuum accumulator or reservoir 20 may be disconnected from the vacuum source 16 or left coupled during use. If disconnected, the medical suction device 10 maintains or stores a negative pressure to at least a portion of the first predetermined vacuum level that may be utilized by the medical and surgical instrument or effector 28. In one embodiment, the vacuum level in the at least one vacuum accumulator or reservoir 20 is the same or less than the first predetermined vacuum level provided from the vacuum source 16. In other embodiments, the vacuum level or negative pressure in the at least one vacuum accumulator or reservoir 20 may be greater than the first predetermined vacuum level. In this regard, the medical suction device 10 may optionally comprise at least one vacuum boosting pump 34 or means or apparatus (such as is shown in the embodiment of FIG. 7), which will be described in more detail later herein, to increase the negative pressure in the at least one vacuum accumulator or reservoir 20 such that the vacuum level in the at least one vacuum accumulator or reservoir 20 is at a second predetermined vacuum level and greater than the first predetermined vacuum level provided from the vacuum source 16. Without a vacuum boost using the at least one vacuum boosting pump 34, the vacuum level (i.e., the second predetermined vacuum level) in the at least one vacuum accumulator or reservoir 20 is the same or less than the first predetermined vacuum level.

Referring back to FIGS. 1-2, the medical suction device 10 may comprise at least one control or control means 38, such as an electronic or manual control that is adapted to control the medical suction device 10, as well as the negative pressure in the at least one outlet port or passageway 26. In this regard, one suitable control may be a foot switch 38a (FIG. 1) which is a manual foot switch that is operatively coupled via a conduit 38b to a solenoid or control valve 40. The at least one control or control means 38 and foot switch 38a causes the solenoid or control valve 40 to open in response to a user, such as a surgeon, operating the foot switch 38a. In response, the solenoid or control valve 40 opens to cause a negative pressure in the at least one outlet port or passageway 26 which is coupled to and in fluid communication with the medical or surgical instrument or effector 28 which is associated with the patient and provides a desired vacuum to the medical and surgical instrument or effector 28. For example, the medical or surgical instrument or effector 28 may be a cautery instrument, the user actuates the foot switch 38a which in turn opens the solenoid or control valve 40 and causes the vacuum to pull debris, vapor, smoke and any fluids and contamination generated during the cautery procedure to be vacuumed into the at least one outlet port or passageway 26. The fluid, debris and the like are then filtered by the at least one filter or filtration means 30 and any fluids are trapped by the at least one fluid trap 32 prior to entering the at least one vacuum accumulator or reservoir 20.

As illustrated by the phantom lines shown in FIG. 5, it is also contemplated that the medical or surgical instrument or effector 28 could have an actuator, actuating means or switch (not shown), such as a hand-operable switch on the medical and surgical instrument or effector 28, for actuating the solenoid or control valve 40, particularly if it is electronic. This embodiment is beneficial when it is desired to have the solenoid or control valve 40 actuated in response to the user using the medical or surgical instrument or effector 28 at the patient site and where the foot switch 38a is not preferred. For example, if the user is a surgeon who is using the medical and surgical instrument or effector 28, such as a cautery device, it is contemplated that the medical or surgical instrument or effector 28 has a switch (not shown) that can be adapted to provide a signal to the solenoid or control valve 40 so that when the user actuates the switch on the medical and surgical instrument or effector 28, it causes the solenoid or control valve 40 to open to provide the negative pressure to the medical and surgical instrument or effector 28. The medical or surgical instrument or effector 28 can be used in conjunction with the foot switch 38a or may be in addition to or separate therefrom. The foot switch 38a, may be wireless, wired, or pneumatic, which may be preferred over electronic coupling due to the fluids that may arise during a surgical procedure.

It should be understood that the medical suction device 10 utilizes the existing vacuum source 16 either with or without the at least one vacuum boosting pump 34. It is important to understand that the at least one coupler 48 and solenoid or control valve 40 are all sized and adapted to control the flow into and out of the at least one vacuum accumulator or reservoir 20. In general, vacuum is delivered in a hospital is delivered to a room at a pressure of about 400 mmHG or about 53 KPa below atmospheric pressure and at a given wall source flow rate, which will depend on hospital the vacuum system design. Typically, it is a very low flow rate.

Regardless, the inventor has found that by providing the at least one vacuum accumulator or reservoir 20, the vacuum can be stored. The at least one coupler 48, conduits, and solenoid or control valve 40 are all sized and adapted to control the flow into and out of the at least one vacuum accumulator or reservoir 20. The sizing of the components will depend, in part, on the vacuum and system of the environment where the medical suction device 10 will be used. It is also important to understand that the flow rate or CFM can be controlled so that a flow rate delivered to the instrument is higher than the wall source flow rate upon demand in response to the user actuating the foot switch 28a. This means that the medical suction device 10 can, for example, provide relatively higher flow rates than that wall source flow rate, which means the user, such as a doctor, may obtain constant or intermittent high flow rates at the surgical site during a surgical procedure, all of which facilitates providing a decontamination system that reduces or eliminates smoke and contamination from the surgical site and around the patient.

Thus, it should be appreciated that the medical suction device 10 comprises the at least one control or control means 38, such as the foot switch 38a or the control associated with the medical and surgical instrument or effector 28, for controlling the operating of the medical suction device 10 and the at least one vacuum accumulator or reservoir 20. The at least one vacuum accumulator or reservoir 20 may comprise a vacuum gauge and control 42 for providing a reading of the vacuum level in the at least one vacuum accumulator or reservoir 20.

Further details of the at least one control or control means 38 are shown in FIG. 5. Although not shown, it is contemplated that the medical suction device 10 may be a “smart” device and comprise wireless means for controlling the various components on the medical suction device 10, such as the check valve or pressure regulator 24, the solenoid or control valve 40, vacuum gauge and control 42, and the medical or surgical instrument or effector 28 and for providing additional information such as the pressure gauged or measured by the vacuum gauge and control 42. The at least one control or control means 44 may comprise conventional wireless communication, WI-FI or other cloud-based communication so that information can be conveyed to a computer or server (not shown) both during and after use. For example, although not shown, a computer and/or monitor may be provided on or associated with the medical suction device 10 or may be remotely therefrom, which receives information from the at least one control or control means 44 to display to a user. It is also contemplated that the at least o control or control means may be coupled to a network that permits control through a device, such as a smartphone, wireless tablet, computer and monitor, touch screen device or the like.

It should be understood that the at least one control or control means 38 is coupled to the foot switch 38a or solenoid or control valve 40 which is situated between the at least one vacuum accumulator or reservoir 20 and the at least one outlet port or passageway 26 and is adapted to open or close in response to the user input as described earlier. It should also be appreciated that the medical suction device 10 may comprise the at least one filter or filtration means 30 and the at least one fluid trap 32 in both the at least one inlet port or passageway 22 and the at least one outlet port or passageway 26 if desired. As mentioned earlier, the user input may come from a manual switch, a current-activated switch or a triggering in response to the medical or surgical instrument or effector 28 or other external equipment, such as external medical equipment or control devices such as a computer, smart device, mobile device or the like.

In one embodiment, the medical or surgical instrument or effector 28 is an external medical equipment device, such as an electrosurgical device or cautery device (not shown). It should also be appreciated that the at least one control or control means 38 may comprise a timer 44a (FIG. 5) that is adapted to provide suction bursts of a user-selected duration and in response to a user actuation of, for example, the foot switch 38a. The at least one control or control means 38 may also comprise or be responsive to a remote or non-remote switch 38c that is under the control of the user as well. In one embodiment, the at least one control or control means 38 may comprise at least one graphical user interface 44c for controlling at least one of suction activation, suction timing, accumulator pressure or pressure inside the at least one vacuum accumulator or reservoir 20, input pressure or output pressure. Thus, the at least one control or control means 38 allows a user to control the airflow and pressure going into and out of the at least one vacuum accumulator or reservoir 20 as well as controlling the pressure inside the at least one vacuum accumulator or reservoir 20. As stated earlier, the at least one coupler 48, conduits, and solenoid or control valve 40 are all sized and adapted to control the flow into and out of the at least one vacuum accumulator or reservoir 20.

Referring now to FIGS. 3-5, further details of various embodiments of the medical suction device 10 will now be described. FIGS. 3-5 show various integral and external components of the medical suction device 10 which include at least one vacuum accumulator or reservoir 20 that is adapted to store a predetermined amount of vacuum and flow rate at the surgical site.

As mentioned earlier, the at least one vacuum accumulator or reservoir 20 is coupled to the at least one inlet port or passageway 22 and at least one outlet port or passageway 26 as shown. As mentioned earlier, the at least one outlet port or passageway 26 comprises the solenoid or control valve 40 which is used in cooperation with the foot switch 38a or other switch, such as a switch on the medical and surgical instrument or effector 28, or other means for controlling the operation of the solenoid or control valve 40 so that it can provide a vacuum or negative pressure to the medical and surgical instrument or effector 28. In this embodiment, the at least one vacuum accumulator or reservoir 20 has a volume of about 1-4 liters, but it should be understood that it could be higher or lower.

In general, the at least one vacuum accumulator or reservoir 20 is conventionally mounted inside the housing 12, but it should be understood that it could be removeable therefrom or the housing 12 and may define the at least one vacuum accumulator or reservoir 20. If housed, the housing 12 may comprises a door (not shown) that permits access to an interior area 12a (FIG. 3) of the housing 12 so that the various components therein may be serviced, changed or accessed if desired.

The medical suction device 10 comprises at least one coupler 48 (FIGS. 2 and 5) for detachably coupling the at least one vacuum accumulator or reservoir 20 to the vacuum source 16 and for providing fluid communication between the at least one vacuum accumulator or reservoir 20 and the vacuum source 16 so that a pressure lower than atmospheric pressure can be maintained in the at least one vacuum accumulator or reservoir 20 when the at least one vacuum accumulator or reservoir 46 is not coupled to the vacuum source 16. It should be appreciated that the medical suction device 10 may be operated as a stand-alone device and may provide a vacuum by storing the at least one vacuum accumulator or reservoir 20, even when it is not coupled to the vacuum source 16. This is advantageous because the medical suction device 10 can be moved anywhere in the room to a desired location where it can be coupled to and provide vacuum to the medical and surgical instrument or effector 28. As mentioned earlier, the at least one coupler 48 is adapted to have a conventional quick change fitting (not shown) that is adapted to be coupled directly to the vacuum source 16 which traditionally is located on a wall in the hospital room or surgical suite.

As also mentioned earlier, the medical suction device 10 comprises the at least one control or control means 38 that comprises at least one coupler or control. In one embodiment, the at least one control or control means 38 comprises a check valve or pressure regulator 24 for controlling the delivery of the negative pressure in the at least one vacuum accumulator or reservoir 20. It should be appreciated that the check valve or pressure regulator 24, coupling 26a and/or at least one coupler 48 are adapted to allow for quick disconnection of the medical suction device 10 from the vacuum source 16 after the at least one vacuum accumulator or reservoir 20 is charged with a vacuum and while retaining the vacuum in the at least one vacuum accumulator or reservoir 20. The at least one coupler 48 allows the medical suction device 10 to be wheeled around and portably transported while holding a vacuum in at least one vacuum accumulator or reservoir 20 in order to enable the medical suction device 10 to supply a vacuum to the medical or surgical instrument or effector 28 without an external connection to the vacuum source 16. Thus, the at least one vacuum accumulator or reservoir 20 is adapted to store a predetermined amount of negative pressure or vacuum after being coupled to and then disconnected from the vacuum source 16, with the at least one vacuum accumulator or reservoir 20 being transportable or moveable in response to movement of the housing 12 utilizing the plurality of wheels 14 which are adapted to support the at least one vacuum accumulator or reservoir 20 and enable it to be moved, portable or transported in the room.

The vacuum source 16 provides a substantially continuous low-flow vacuum to the at least one vacuum accumulator or reservoir 20. As best illustrated in FIGS. 3 and 5 and as described earlier herein, the medical suction device 10 may comprise a at least one vacuum boosting pump 34 (FIG. 5) and be electronic or mechanical, such as a manual foot pump (not shown) that is coupled to the at least one vacuum accumulator or reservoir 20 for boosting the vacuum in the at least one accumulator or reservoir 20. Although not shown, the at least one vacuum boosting pump 34 may be provided with a coupling (not shown) that is adapted to fit the at least one coupler 48 or otherwise be coupled into the at least one inlet port or passageway 22 on the at least one vacuum accumulator or reservoir 20. Alternatively, it could be separately and detachably coupled to and in fluid communication with the at least one vacuum accumulator or reservoir 20 as illustrated in FIG. 5. As mentioned, the at least one vacuum boosting pump 34 could be a manual pump, a foot actuated pump, a mechanical, pneumatic or hydraulic pump or an electric pump adapted to increase a negative pressure in the at least one vacuum accumulator or reservoir 20 in response to, for example, a user actuating the at least one vacuum boosting pump 34. Alternatively, the at least one vacuum boosting pump 34 could be provided that is electronic that is either battery operated or coupled to a conventional power outlet or source so that when it is turned on, it facilitates increasing the negative pressure in the at least one vacuum accumulator or reservoir 20.

Thus, the medical suction device 10 may comprise the at least one vacuum boosting pump 34 for the medical suction device 10 to be used without an external vacuum connection through the vacuum source 16 or when the vacuum source 16 is not sufficient to generate the desired vacuum levels in the at least one vacuum accumulator or reservoir 20. Again, the at least one vacuum boosting pump 34 may be used separate from, in addition to or in combination with the vacuum source 16 so that the second predetermined vacuum level inside the at least one vacuum accumulator or reservoir 20 may be increased to above both atmospheric pressure and the first predetermined vacuum level provided by the vacuum source 16.

The at least one vacuum boosting pump 34 may be operatively situated inside the housing 12 or it may be a separate component and situated on or outside of the housing 12 for creating or increasing a negative pressure in the at least one vacuum accumulator or reservoir 20. In this regard, the medical suction device 10, therefore, provides means and apparatus for evacuating fluid, means for increasing a vacuum in the at least one vacuum accumulator or reservoir 20, even when there is no vacuum source 16 or when the vacuum provided at the vacuum source 16 is less than a predetermined level. This embodiment is also advantageous for rooms that do not have any vacuum source 16 so that the user can rely on the at least one vacuum boosting pump 34 to evacuate the at least one vacuum accumulator or reservoir 20 by the predetermined vacuum level which, again, may be greater than atmospheric pressure as well as the first predetermined vacuum level.

In order to judge or gauge the amount of negative pressure in the at least one vacuum accumulator or reservoir 20, the vacuum gauge and control 42 is provided and is coupled to and in fluid communication with the at least one vacuum accumulator or reservoir 20 and under control of the at least one control or control means 38. The vacuum gauge or control 42 enables a user to determine the amount of vacuum, particularly negative pressure, in the at least one vacuum accumulator or reservoir 20.

FIG. 7 illustrates another embodiment and means for increasing a vacuum pressure and/or capacity in the at least one vacuum accumulator or reservoir 20. In this embodiment, the at least one vacuum accumulator or reservoir 20 comprises a hydraulic cylinder or solenoid 50 having an armature 52 coupled to a piston 56 as shown. The piston 56 comprises at least one or a plurality of seals or rings (not shown) to provide an airtight junction between a wall 56a of the piston 56 and a wall 20a of the at least one vacuum accumulator or reservoir 20. The hydraulic cylinder or solenoid 50 is a mechanical or electronic solenoid and actuates the piston 56 to increase or decrease a volume in the area 60 of the at least one vacuum accumulator or reservoir 20 in response to a user input. The piston 56 is moved in the direction of arrow A in FIG. 7 to decrease the volume in area 60 by moving the piston 56 in the direction of arrow A. The piston 56 is caused to move in an opposite direction of arrow B to increase the volume in area 60. Increasing the volume in area 60 causes a resultant increase in vacuum in direct proportion to the increase in volume in accordance with Boyle's law. By altering the position of the piston 56, the volume in area 60 inside the at least one vacuum accumulator or reservoir 20 and the negative pressure therein may be increased or decreased without regard to the amount of vacuum or negative pressure provided by either the vacuum source 16 or the at least one vacuum boosting pump 34. By moving the piston 56, a pressure differential is created in the areas 60a and 60b and the vacuum or negative pressure in the closed system in the at least one vacuum accumulator or reservoir 20 and in the area 60 may be increased by a downward movement in the direction of arrow B of the piston 56 or it may be decreased by movement in the direction of arrow A in FIG. 7.

Thus, the piston 56 and the hydraulic cylinder or solenoid 50 that actuates the piston 56 increases or decreases the volume in the at least one vacuum accumulator or reservoir 20 in order to increase or decrease a negative pressure in the at least one vacuum accumulator or reservoir 20. Advantageously, the at least one vacuum boosting pump 34, the piston 56, the hydraulic cylinder or solenoid 50 and the like provide means for increasing the amount of vacuum or negative pressure in the at least one vacuum accumulator or reservoir 20.

Both of the hydraulic cylinder or solenoid 50 and the at least one vacuum boosting pump 34 may be separately controlled by the user via at least one control or control means 38 which may include the at least one graphical user interface 44c, for example, adapted to permit the user to not only see the vacuum level inside the at least one vacuum accumulator or reservoir 20, but also to adjust it using one or both of the at least one vacuum boosting pump 34 and/or the piston 56. The vacuum gauge and control 42 may also be used.

In the illustration being described, the at least one vacuum accumulator or reservoir 20 comprises at least one leak resistant container of metallic or polymer construction such that there is a fixed pressure and volume relationship within the at least one vacuum accumulator or reservoir 20. In one embodiment the relationship can be 1:1. The at least one vacuum accumulator or reservoir 20 may comprise at least one of a cylinder or hollow body adapted to define the area 60 or it may be segregated into different areas, such as areas 60a and 60b inside the at least one vacuum accumulator or reservoir 20.

In the illustration being described, the at least one vacuum accumulator or reservoir 20 comprises a leak-resistant, variable-volume container and reservoir adapted to define a predetermined pressure-volume relationship in the at least one vacuum accumulator or reservoir 20. In the illustrating being described, the leak-resistant, variable-volume container and reservoir comprises at least one of a flexible membrane, a flexible bladder, a moveable piston or other means or apparatus to vary a volume within the area 60 of the at least one vacuum accumulator or reservoir 20 at a third predetermined vacuum level which can be greater or less than the first and second predetermined vacuum levels, but is preferably greater in the illustrating being described. It should also be appreciated that the first, second and third predetermined vacuum levels are also preferably greater than atmospheric pressure surrounding the medical suction device 10. In one illustrative embodiment, the third predetermined vacuum level is greater than both the first and second predetermined vacuum levels.

Returning to FIGS. 1-5, it should be appreciated that the medical suction device 10 comprises at least one decontamination means or decontaminator 31 may comprise the at least one filter or filtration means 30 for decontaminating any fluid or air stream entering into the at least one outlet port or passageway 26 and prior to entering the at least one vacuum accumulator or reservoir 20. The at least one decontamination means or decontaminator 31 facilitates decontaminating or maintaining a decontaminated at least one vacuum accumulator or reservoir 20 in order to prevent survival and growth of microorganisms, bacteria, unwanted contamination and the like within the at least one vacuum accumulator or reservoir 20. In one embodiment, the at least one decontamination means or decontaminator 31 comprises the at least one filter or filtration means 30 for removing contamination and organisms into or out of the at least one vacuum accumulator or reservoir 20. In the illustrating being described, at least one decontamination means or decontaminator 31 and the at least one filter or filtration means 30 comprises at least one of mechanical, absorptive, catalytic or chemical filtration capacities, including ULPA (ultra-low penetration air) filtration, HEPA (high efficiency particulate air) filtration, carbon filtration, catalytic filtration, pleated media filtration, solid media filtration, ultraviolet germicidal irradiation or other means for removing airborne contamination and organisms.

Advantageously, the at least one fluid trap 32 is in fluid communication with the at least one outlet port or passageway 26 and the at least one filter or filtration means 30 cooperate to facilitate causing the medical suction device 10 to become or maintain a dry vacuum so that the at least one vacuum accumulator or reservoir 20 does not collect fluids and contamination when the medical or surgical instrument or effector 28 is being used by the user. In the illustration being described, the at least one fluid trap 32 is situated between the at least one vacuum accumulator or reservoir 20 and the medical or surgical instrument or effector 28 in order to trap fluids prior to entering or leaving the medical suction device 10 so that the at least one vacuum accumulator or reservoir 20 remains contamination free is dry and adapted to define a dry vacuum accumulator. Advantageously, the medical or surgical instrument or effector 28 collects or generates vapors or fluids during a medical procedure and the at least one vacuum accumulator or reservoir 20 is adapted to provide vacuum to the medical or surgical instrument or effector 28 in order to collect fluids, contamination, smoke, vapor and/or other debris and the like during use of the medical and surgical instrument or effector 28. As mentioned, the at least one fluid trap 32 maintains the dry environment within the at least one vacuum accumulator or reservoir 20 by trapping fluids, smoke, vapor and/or debris and the like by preventing it from entering into the at least one vacuum accumulator or reservoir 20.

It should be understood that the medical suction device 10 may comprise at least one or a plurality of valves, such as the check valve or pressure regulator 24, vacuum gauge and control 42, solenoid or control valve 40 and the like, that cooperate and are adapted to control the vacuum level in the at least one vacuum accumulator or reservoir 20. The medical suction device 10 may also comprise at least one pressure sensor 64 (FIG. 5) coupled to the at least one control or control means 38. The at least one pressure sensor 64 is in fluid communication with the at least one vacuum accumulator or reservoir 20 for sensing a pressure in the at least one vacuum accumulator or reservoir 20.

The medical suction device 10 may further comprise at least one velocity sensor 66 (FIG. 5) coupled to either the at least one outlet port or passageway 26 for sensing a velocity of airflow in the at least one inlet port or passageway 22 and the at least one outlet port or passageway 26 and into or out of the at least one vacuum accumulator or reservoir 20.

Advantageously, the medical suction device 10 is a dry accumulator that is portable while not collecting fluids in the at least one vacuum accumulator or reservoir 20 during the use of the medical and surgical instrument or effector 28. During use, the at least one vacuum accumulator or reservoir 20 can be initially charged by the vacuum source 16 and then disconnected therefrom so that the check valve or pressure regulator 24 enables the at least one vacuum accumulator or reservoir 20 to maintain and hold the vacuum achieved. The vacuum may be boosted by the at least one vacuum boosting pump 34 if desired or by altering the position of the piston 56 in the embodiment of FIG. 7 as mentioned earlier. The medical suction device 10 can be moved to a desired location after the vacuum in the at least one vacuum accumulator or reservoir 20 has achieved the desired second predetermined vacuum level. The medical suction device 10 may be moved in proximity to a patient, where the user may actuate the foot switch 38a or other means to activate the vacuum and provide the vacuum to the medical or surgical instrument or effector 28 during a procedure. The medical suction device 10 may be located proximate to the patient, but it could also be located remote from the patient, especially if the activation of the medical suction device 10 is in response to the activation of the medical or surgical instrument or effector 28 which is in fluid communication with the at least one outlet port or passageway 26 and remote therefrom. Likewise, the foot switch 38a could be located proximate to the user while the at least one vacuum accumulator or reservoir 20 is located remote from the user. In this regard, the foot switch 38a may be pneumatically or electronically coupled to the solenoid or control valve 40 via the conduit 38b, but it is contemplated that the foot switch 38a could be wireless as well so that the foot switch 38a can operate the solenoid or control valve 40 remotely and without a physical coupling.

FIG. 6 is a general schematic diagram or flow chart of a procedure in accordance with one embodiment of the invention. The procedure begins at block 70 where the medical suction device 10 or the at least one vacuum accumulator or reservoir 20 is connected to the vacuum source 16 whereupon the at least one vacuum accumulator or reservoir 20 is evacuated so that air is removed from the at least one vacuum accumulator or reservoir 20 and it is charged with a vacuum (step 72 in FIG. 6). After the at least one vacuum accumulator or reservoir 20 is charged to the second predetermined level with a vacuum, it can be optionally disconnected from the vacuum source 16 or it can remain connected thereto. The user may then position or move the medical suction device 10 to a desired location and couple the medical or surgical instrument or effector 28 to the coupling 26a and the at least one outlet port or passageway 26. When desired, the user can actuate the solenoid or control valve 40, such as by use of the foot switch 38a or by actuating a switch (not shown) on the medical or surgical instrument or effector 28 for those embodiments that provide the vacuum in response to the user actuating or energizing the medical and surgical instrument or effector 28. During use of the medical suction device 10, the user actuates the solenoid or control valve 40 selectively upon vacuum need during use of the medical and surgical instrument or effector 28. It should be appreciated that the user may use the medical or surgical instrument or effector 28 and intermittently actuate the solenoid or control valve 40 in order to provide either a vacuum or a vacuum boost to the medical and surgical instrument or effector 28. It should be understood that vacuum does not have to be provided at all times when the medical or surgical instrument or effector 28 is being used. The user can selectively and intermittently provide vacuum to the medical or surgical instrument or effector 28 by actuating the foot switch 38a or the switch on the medical and surgical instrument or effector 28. Thus, although not shown, it is contemplated that the medical or surgical instrument or effector 28 itself may comprise a switch or activation means or actuator for actuating the solenoid or control valve 40 either directly or remotely during use of the medical or surgical instrument or effector 28 when the vacuum is desired. Like the foot switch 38a, the medical or surgical instrument or effector 28 may also be adapted to wirelessly control the solenoid or control valve 40.

At block 74, the user actuates the release or solenoid or control valve 40 on the medical suction device 10 to selectively provide vacuum upon need. At block 76 in FIG. 6, the user can create an intermittent airflow into the at least one vacuum accumulator or reservoir 20 of higher flow than the vacuum source 16, particularly when the vacuum in the at least one vacuum accumulator or reservoir 20 has been negatively pressured such that the second predetermined vacuum level is higher than the first predetermined vacuum level. In these embodiments, the user can then direct intermittent high-flow vacuum to the medical or surgical instrument or effector 28 and the patient at block 78.

### ADDITIONAL CONSIDERATIONS

1. Advantageously, the medical suction device 10 is adapted to effectively increase the airflow and negative pressure arising from existing wall suction systems. Because suction needs are typically intermittent, the continuous vacuum provided by wall suction can be stored in the at least one vacuum accumulator or reservoir 20 which will generate a progressively negative pressure or vacuum while not in use. The medical suction device 10 will further incorporate the valve means or solenoid or control valve 40 whereby a user can generate intermittent bursts to the medical or surgical instrument or effector 28 of high vacuum flows via the at least one vacuum accumulator or reservoir 20. The medical suction device 10 further comprises various filtration, control, and safety means described earlier.

2. The medical suction device 10 is therefore adapted to provide at least one vacuum accumulator or reservoir 20 that is adapted to provide continuous or intermittent vacuum to the instrument 20 during use by a user. As mentioned, the medical suction device 10 can be actuated by the foot switch 38a or other at least one control or control means 38 for controlling the medical suction device 10 such as those mentioned earlier wherein the medical or surgical instrument or effector 28 itself comprises the at least one control or control means 38 for actuating the solenoid or control valve 40 in order to deliver vacuum to the medical or surgical instrument or effector 28 and patient.

3. In order to activate the medical suction device 10, the user can utilize the foot switch 38a, a manual switch, a current-activated switch or a triggering from the external medical equipment or the medical and surgical instrument or effector 28, such as an electrosurgery device, cautery device or the like as mentioned earlier. As mentioned previously, the solenoid or control valve 40 or the medical suction device 10 may optionally include the timer or timer means 44a (FIG. 5) to provide suction bursts of a user-selected duration. It is contemplated that the bursts can be intermittent, continuous, or even of predetermined duration, such as continuous for a predetermined time of 2-10 seconds or longer. Thus, a timeout can be provided after a certain period of time, such as 3 seconds from actuation of the foot switch 38a.

4. As is known, vacuum levels refer to the numerical measurement of vacuum pressure using a unit of measurement scale that quantifies how much lower the pressure is compared to atmospheric pressure. These are typically measured in Hg (mmHg), Torr (mmHg), mbar (a), Pa (%), and other units of measurement, such as CFM and PSI. When referring herein to the second predetermined vacuum level in the at least one vacuum accumulator or reservoir 20 being greater (i.e., more negative) than the negative pressure of the first predetermined vacuum level from the input source 16. Consequently, the negative pressure of the first predetermined vacuum level is lower (i.e., more negative) than atmospheric pressure compared to the negative pressure of the first predetermined vacuum, which is greater (i.e., less negative). Consequently, the at least one vacuum accumulator or reservoir 20 is capable of increasing the vacuum or making pressure more negative compared to the negative pressure or vacuum provided by the input source. If the negative pressure of the second predetermined vacuum level is less negative than the first predetermined vacuum level, then the vacuum boost pump or the embodiment of FIG. 7 may be utilized if desired to increase the negative pressure (i.e., make it more negative) so that the pressure level becomes farther below atmospheric pressure than the negative pressure associated with the first predetermined vacuum level. Throughout the disclosure, references to increasing the negative pressure or causing the second predetermined pressure level of the at least one vacuum accumulator or reservoir 20 to be greater than the first predetermined vacuum level from the input source 16 means that negative pressure is increasing and going farther below atmospheric pressure compared to the negative pressure associated with the first predetermined vacuum level.

However, even without an increase of the negative pressure, in all embodiments, the components of the medical suction device 10, the medical or surgical instrument or effector 28, at least one coupler 48, the solenoid or control valve 40 are sized and adapted so that during use, the flow rate at the medical or surgical instrument or effector 28 and eventually into the at least one vacuum accumulator or reservoir 20 is increased to a higher flow rate (CFM) than the flow rate provided by the vacuum source 16, thereby enabling the user, such as a surgeon, to use the foot switch 38a to intermittent or continuous utilize the higher air flow at the surgical site, This, in turn, facilitates eliminating or reducing infectious aerosols and surgical smoke, fluids and debris from surgical site. Thus, the medical suction device 10 is capable of providing a higher negative pressure (i.e., farther below atmospheric pressure) as well as the ability to increase the flow rate at the medical or surgical instrument or effector 28. As mentioned herein, flow can be on demand, intermittent or even timed in response to the user using the foot switch 38a.

Advantageously, another embodiment of this invention, including all embodiments shown and described herein, could be used alone or together and/or in combination with one or more of the features covered by one or more of the claims set forth herein, including but not limited to one or more of the features or steps mentioned in the Summary of the Invention and the claims.

While the system, device and method herein described constitute preferred embodiments of this invention, it is to be understood that the invention is not limited to this precise system, apparatus and method, and that changes may be made therein without departing from the scope of the invention which is defined in the appended claims.

What is claimed is:

1. A medical suction device for use in a room, said medical suction device comprising:
a vacuum accumulator that defines at least one vacuum accumulator or reservoir in fluid communication with at least one inlet port for coupling to a vacuum source and at least one outlet port for coupling to an instrument that utilizes a vacuum, said vacuum source providing a vacuum at a first predetermined vacuum level;
at least one control or control means for controlling an operation of said medical suction device;
said medical suction device being adapted to store a vacuum charge in said at least one vacuum accumulator or reservoir so that said at least one vacuum accumulator or reservoir may be disconnected from said vacuum source and still maintain at least a portion of said first predetermined vacuum level which may be utilized by said instrument.

2. The medical suction device as recited in claim 1 wherein said at least one vacuum accumulator or reservoir is portable and moveable when said at least one inlet port is coupled to said vacuum source and when it is disconnected therefrom while maintaining a vacuum at a second predetermined vacuum level.

3. The medical suction device as recited in claim 2 wherein said second predetermined vacuum level is less negative than said first predetermined vacuum level.

4. The medical suction device as recited in claim 2 wherein said second predetermined vacuum level is more negative than said first predetermined vacuum level.

5. The medical suction device as recited in claim 1 wherein an airflow rate at the at least one outlet port is greater than an airflow rate at said at least one inlet.

6. The medical suction device as recited in claim 4 wherein an airflow rate at the at least one outlet port is greater than an airflow rate at said at least one inlet.

7. The medical suction device as recited in claim 2 wherein said first predetermined vacuum level is substantially the same as or less negative than said second predetermined vacuum level while the airflow rate at the instrument is greater than an airflow rate at the at least one outlet port.

8. The medical suction device as recited in claim 2 wherein said first predetermined vacuum level is different than said second predetermined vacuum level while the airflow rate at the instrument is greater than an airflow rate at the at least one outlet port.

9. The medical suction device as recited in claim 1 wherein said at least one control means or control comprises at least one of a switch, a foot switch or actuator, the airflow rate being constant, intermittent or timed out in response to a user actuating the at least one switch, foot switch or actuator.

10. The medical suction device as recited in claim 1 wherein said medical suction device comprises at least one coupler for detachably coupling said at least one vacuum accumulator or reservoir to said vacuum source and providing fluid communication between said at least one vacuum accumulator or reservoir and said vacuum source so that a pressure lower than atmospheric pressure can be maintained in said at least one vacuum accumulator or reservoir when said at least one vacuum accumulator or reservoir is not coupled to said vacuum source.

11. The medical suction device as recited in claim 10 wherein said at least one coupler comprises at least one of a check valve or pressure regulator.

12. The medical suction device as recited in claim 10 wherein said at least one coupler is adapted to allow for disconnection of said medical suction device from said vacuum source after said at least one vacuum accumulator or reservoir is charged with a vacuum and while retaining said vacuum in said at least one vacuum accumulator or reservoir, said at least one coupler allowing said medical suction device to be portably transported while holding a vacuum in said at least one vacuum accumulator or reservoir in order to enable said medical suction device to supply a vacuum without an external connection to said vacuum source.

13. The medical suction device as recited in claim 1 wherein said at least one vacuum accumulator or reservoir is adapted to store a predetermined amount of negative pressure after being coupled to and then disconnected from said vacuum source, said at least one vacuum accumulator or reservoir being transportable.

14. The medical suction device as recited in claim 11 wherein said at least one vacuum accumulator or reservoir comprises a plurality of wheels for supporting said at least one vacuum accumulator or reservoir and for enabling it to be moved, portable or transported in the room.

15. The medical suction device as recited in claim 1 wherein said vacuum source provides a substantially continuous low-flow vacuum to said at least one vacuum accumulator or reservoir, said medical suction device comprising at least one vacuum booster for boosting said substantially continuous low-flow vacuum from said first predetermined vacuum level to a second predetermined vacuum level, said first predetermined vacuum level being lower than said second predetermined vacuum level.

16. The medical suction device as recited in claim 15 wherein said at least one vacuum booster further comprises an internal powered or manual vacuum pump for the medical suction device to be used without an external vacuum connection to said vacuum source or when said vacuum source is not sufficient to generate desired vacuum levels in said at least one vacuum accumulator or reservoir.

17. The medical suction device as recited in claim 15 wherein said at least one vacuum booster comprises a pump operatively situated in or on said medical suction device for creating or increasing a negative pressure in said at least one vacuum accumulator or reservoir.

18. The medical suction device as recited in claim 15 wherein said at least one vacuum booster comprises a piston coupled to a piston drive for driving said piston inside said at least one vacuum accumulator or reservoir, said piston being driven by said piston drive in order to change a volume in said at least one vacuum accumulator or reservoir in order to increase a negative pressure in said at least one vacuum accumulator or reservoir.

19. The medical suction device as recited in claim 18 wherein said at least one vacuum booster comprises a pump operatively coupled to said at least one vacuum accumulator or reservoir for increasing a negative pressure in said at least one vacuum accumulator or reservoir.

20. The medical suction device as recited in claim 2 wherein said vacuum source is external to said medical suction device and said vacuum charge from said vacuum source creates a contained pressure environment at said second predetermined level in said at least one vacuum accumulator or reservoir that is lower than atmospheric pressure, said at least one vacuum accumulator or reservoir comprising said at least one inlet port for coupling to said vacuum source and said at least one outlet port for coupling for supplying vacuum to said instrument.

21. The medical suction device as recited in claim 1 wherein said at least one vacuum accumulator or reservoir comprises at least one leak resistant container of metallic or polymer construction such that there is a fixed pressure and volume relationship within said at least one vacuum accumulator or reservoir, said at least one vacuum accumulator or reservoir comprising at least one of a cylinder or hollow body adapted to define said at least one vacuum accumulator or reservoir.

22. The medical suction device as recited in claim 1 wherein said at least one vacuum accumulator or reservoir comprises a leak-resistant, variable-volume container adapted to define a predetermined pressure-volume relationship in said at least one vacuum accumulator or reservoir.

23. The medical suction device as recited in claim 22 wherein said leak-resistant, variable-volume container comprises at least one of a flexible membrane, a flexible bladder, or a moveable piston adapted to vary a volume within said at least one vacuum accumulator or reservoir while retaining a vacuum in said at least one vacuum accumulator or reservoir at a third predetermined vacuum level.

24. The medical suction device as recited in claim 23 wherein said third predetermined vacuum level is greater than both said first predetermined vacuum level and said second predetermined vacuum level.

25. The medical suction device as recited in claim 1 wherein said medical suction device comprises at least one decontamination means or decontaminator for decontaminating to facilitate decontaminating said at least one vacuum accumulator or reservoir in order to prevent survival and growth of microorganisms within said at least one vacuum accumulator or reservoir or said medical suction device.

26. The medical suction device as recited in claim 1 wherein said at least one control or control means comprises a switch having at least one valve situated between said at least one vacuum accumulator or reservoir and said at least one inlet port and adapted to open or close in response to a user input.

27. The medical suction device as recited in claim 26 wherein said user input may comprise a foot pedal, manual switch, current-activated switch or a triggering in response to said instrument or external medical equipment.

28. The medical suction device as recited in claim 27 wherein said external medical equipment is an electrosurgery device.

29. The medical suction device as recited in claim 21 wherein said at least one control or control means comprises timer to provide suction bursts of a user-selected duration.

30. The medical suction device as recited in claim 1 wherein said at least one outlet port is adapted and dimensioned to receive various medical and surgical effector couplings, said medical and surgical effector couplings comprising at least one of a medical tube, a suction tip, an electrosurgical pencil, a smoke evacuator device, a laparoscopic device, a cautery or a surgical device that requires operable suction.

31. The medical suction device as recited in claim 1 wherein said at least one control or control means comprises at least one user interface for controlling at least one of suction activation, suction timing, accumulator pressure, input pressure or output pressure.

32. The medical suction device as recited in claim 1 wherein said medical suction device comprises at least one of at least one valve for controlling a vacuum level in said at least one vacuum accumulator or reservoir, at least one pressure sensor for sensing a pressure in said at least one vacuum accumulator or reservoir, or at least one velocity sensor for sensing a velocity of airflow into or out of said at least one vacuum accumulator or reservoir.

33. The medical suction device as recited in claim 1 wherein said medical suction device further comprises:
at least one filter or filter filtration means for removing contamination and organisms into or out of said at least one vacuum accumulator or reservoir from an airflow.

34. The medical suction device as recited in claim 33 wherein said at least one filter or filter filtration means comprises at least one of mechanical, absorptive, catalytic or chemical filtration capacities, including ULPA (ultra-low penetration air) filtration, HEPA (high efficiency particulate air) filtration, carbon filtration, catalytic filtration, pleated media filtration, solid media filtration, ultraviolet germicidal irradiation, and similar means for removing airborne contamination and organisms.

35. The medical suction device as recited in claim 1 wherein said at least one vacuum accumulator or reservoir is a dry vacuum and does not collect fluids when said instrument is being used by a user.

36. The medical suction device as recited in claim 1 wherein said medical suction device comprises a fluid trap situated between said at least one vacuum accumulator or reservoir and said instrument in order to trap fluids prior to entering said medical suction device so that said at least one vacuum accumulator or reservoir is a dry vacuum accumulator.

37. The medical suction device as recited in claim 1 wherein said instrument collects or generates vapor or fluids during a medical procedure and in said at least one vacuum accumulator or reservoir and provides said vacuum to said instrument in order to collect fluids, smoke, vapor and/or debris and the like during the medical procedure; said medical suction device further comprising a fluid trap for preventing said fluids, smoke, vapor and/or debris or contamination from entering said at least one vacuum accumulator or reservoir.

38. A medical suction device comprising:
an external connection to a vacuum source;
a vacuum accumulator;
a user selectable suction switch;
at least one suction output port; and
a control means;
wherein said medical suction device receives a continuous low-flow vacuum input and transmits an intermittent high-flow vacuum output;
said intermittent high-flow vacuum output initiated by user activation of said switch.

39. The medical suction device as recited in claim 38 wherein said external connection comprises a hose or piping means to attach to an external vacuum source;
said external connection communicates an external vacuum charge with said vacuum accumulator.

40. The medical suction device as recited in claim 38 wherein said external connection means further comprising a one way check valve and/or pressure regulator;
said check valve to allow for disconnection of said suction device from said vacuum source after said vacuum charge while retaining internal accumulator vacuum;
said disconnection and retention allowing said suction device to be portably transported and used without an external connection.

41. The medical suction device as recited in claim 38 wherein said vacuum accumulator receiving a vacuum charge from said external connection to create a contained pressure environment lower than atmospheric pressure;
said accumulator comprising a vacuum input and output means.

42. The medical suction device as recited in claim 38 wherein said vacuum accumulator comprising a rigid, leak-resistant container of metallic or polymer construction such that there is a fixed pressure/volume relationship within said accumulator;
said accumulator comprising a cylinder or other hollow body.

43. The medical suction device as recited in claim 38 wherein said vacuum accumulator comprising a non-rigid, leak-resistant, variable-volume container of metallic or polymer construction such that there is a variable pressure/volume relationship within said accumulator;
said variable-volume means comprising flexible membrane, mobile piston or similar means to vary accumulator volume, while retaining a vacuum seal;
said variable-volume means providing a benefit of increased vacuum capacity and/or negative pressure levels.

44. The medical suction device as recited in claim 38 wherein said vacuum accumulator further comprising an internal decontamination means consisting of ultraviolet irradiation, catalytic oxidation, electrostatic generation, ion generation, ozone generation, or similar means to prevent survival and growth of microorganisms within the accumulator and or suction device.

45. The medical suction device as recited in claim 38 wherein said suction switch comprising a valve between said accumulator and said at least one suction port;
said valve comprising an electromagnetic solenoid or similar mechanically actuated design, providing near instantaneous opening of said valve in response to a user input;
said input may comprise a foot pedal, manual switch, current-actuated switch, or triggering from external medical equipment, such as an electrosurgery device;
said valve may optionally include a timer means to provide suction bursts of a user-selected duration.

46. The medical suction device as recited in claim 38 wherein said at least one suction port dimensioned to receive various medical and surgical effector ends such as medical tubing, suction tips, electrosurgical pencils, smoke evacuation devices, laparoscopic devices, and other means which require operable suction.

47. The medical suction device as recited in claim 38 wherein said control means comprising a user interface controlling one or more of suction activation, suction timing, accumulator pressure, input pressure and output pressure;
said interface in communication with corresponding valves, pressure, and/or velocity sensors.

48. The medical suction device as recited in claim 38 wherein said medical suction device and/or said vacuum accumulator comprising a filtration means, with at least one of mechanical, absorptive, catalytic or chemical filtration capacities, including ULPA filtration, HEPA filtration, carbon filtration, catalytic filtration, pleated media filtration, solid media filtration, ultraviolet germicidal irradiation, and similar means for removing airborne contamination and organisms.

49. The medical suction device as recited in claim 38 wherein said medical suction device optionally comprising an internal powered or manual vacuum pump to allow for said device to be used without an external vacuum connection, or when said external vacuum source is not sufficient to generate adequate accumulator vacuum levels.

50. A method for providing medical suction comprising:
connecting an accumulator to a constant low-flow vacuum source;
evacuating air from said accumulator;
selectively actuating a release valve on said accumulator thus creating an intermittent vacuum-powered airflow of greater volume (for instance as measured in cubic feet per minute) than a constant low-flow volume arising from said vacuum source; and
directing an intermittent high-flow vacuum to an area of need.
